(19) United States
(12) Patent Application Publication
(10) Pub. No.: US 2024/0197164 A1
(43) Pub. Date: Jun. 20, 2024

(54) MEDICAL DEVICE

(71) Applicant: Magnext Life Science Pte. Ltd, Kaki Bukit (SG)

(72) Inventors: Clement Coletto, Ivano- Frankivsk (UA); Sebastien Roeskay, Segny (FR)

(73) Assignee: Magnext Life Science Pte. Ltd, Kaki Bukit (SG)

(21) Appl. No.: 18/290,750

(22) PCT Filed: Oct. 20, 2021
(86) PCT No.: PCT/SG2021/050632

§ 371 Date: Jan. 19, 2024

(30) Foreign Application Priority Data
Oct. 21, 2020 (SG) 10202010446X

(51) Int. Cl. A61B 1/303; A61B 1/00

(52) U.S. Cl. CPC A61B 1/303; A61B 1/00016; A61B 1/00048; A61B 1/00148

(57) ABSTRACT

Disclosed is a medical device. The medical device comprises an elongate body having a proximal region positioned, in use, outside a body cavity, and a distal region positioned, in use, inside the body cavity, an imaging device at the distal region, an anchoring device having two opposed ends and being movable between a collapsed condition to facilitate entry of the imaging device into the body cavity and an expanded condition for engaging an internal wall of the body cavity, and a slider mechanism extending from the proximal region to the anchoring device and being movable to cause relative movement between the two opposed ends of the anchoring device, thereby to move the anchoring device between the collapsed condition and expanded condition. Also disclosed is an imaging system including such a medical device, and a docking station or housing. The medical device may be a colposcope, proctoscope, rhinoscope or other device configured to examine part of the body of a subject.

### TECHNICAL FIELD

The present invention relates, in general terms, to a medical device for imaging an internal region of a body cavity. More particularly, the present invention relates to, but is not limited to, a colposcope.

### BACKGROUND

It has been demonstrated that early detection and treatment of oral and cervical cancers significantly improve survival rates. Detection of precancerous and cancerous lesions is mostly accomplished through visual inspection followed by a biopsy of relevant tissue sites.

For cervical cancer screening, the Papanicolau test (Pap smear) is the standard method of assessment. If the Pap smear is positive, colposcopy and biopsy are performed. Colposcopy is performed using a colposcope which is a device for imaging a cervical region of a subject.

In some countries, access to clinics for colposcopy assessments is difficult. There is therefore a need for a colposcope that can reduce the time spent in a clinic for a colposcopy assessment, or facilitate in-home performance of that assessment.

Existing colposcopes can be difficult to accurately position to enable a camera of the colposcope to clearly view the cervix, particularly when a user is endeavouring to perform an assessment of themselves. It is therefore desirable to provide a colposcope that assists with positioning the camera relative to the cervix.

To properly position the camera, some colposcopes have an enlarged distal end for abutting the cervix. That distal end maintains spacing between the camera and cervix. However, inserting the enlarged distal end into the vision can be difficult and uncomfortable. It is therefore desirable to provide a colposcope that facilitates easy insertion into the vagina.

### SUMMARY

Disclosed herein is a medical device comprising:
an elongate body having a proximal region positioned, in use, outside a body cavity, and a distal region positioned, in use, inside the body cavity;
an imaging device at the distal region;
an anchoring device having two opposed ends and being movable between a collapsed condition to facilitate entry of the imaging device into the body cavity and an expanded condition for engaging an internal wall of the body cavity; and
a slider mechanism extending from the proximal region to the anchoring device and being movable to cause relative movement between the two opposed ends of the anchoring device, thereby to move the anchoring device between the collapsed condition and expanded condition.

The medical device may be a colposcope.

The slider mechanism may comprise an actuator at the proximal region.

The two opposed ends of the anchoring device may a proximal end and a distal end, the distal end being attached at the distal region to one of the elongate body and imaging device. The proximal end may be movable towards the distal end to move the anchoring device to the expanded condition. When the anchoring device is in the expanded condition, the proximal end may be movable away from distal end to move the anchoring device to the collapsed condition.

The anchoring device may comprise a sheath. The sheath may extend from the distal region in a proximal direction.

The sheath may be formed from a piece of material and have a length, and when a distance between the two opposed ends is less than the length, the sheath bends between the two opposed ends under stresses created in the piece of material.

The anchoring device may be configured to expand by bending in a mid-portion between the two opposed ends. When the anchoring device moves from the collapsed condition to the expanded condition, the mid-portion may move from a position proximal of a distal tip of the body to a position distal of the distal tip.

The body may have an axis extending between the proximal region and the distal region, and the anchoring device may be expanded laterally relative to the axis in the expanded condition. The anchoring device may have a plurality of expanded conditions each having a different amount of lateral expansion relative to the axis. The plurality of expanded conditions may be discrete or continuous.

The medical device may comprise a transmitter in communication with the imaging device, for transmitting images captured by the imaging device to a remote system.

Also disclosed herein is a system for imaging a body cavity, comprising:
a medical device as described above;
a housing;
a receiver at the housing; and
a power source for powering the receiver,
   wherein, in use, the transmitter transmits images to the receiver.

In use, the medical device and housing may be physically separate, and the transmitter wirelessly may transmit images to the receiver.

The housing may comprise a dock for engaging a portable device, the receiver relaying the images to the portable device.

Advantageously, the anchoring device as a collapsed condition to facilitate entry into the body cavity. Conversely, the anchoring device also has an expanded condition to facilitate engagement (e.g. contact or abutment) with the internal wall of the body cavity to maintain the position of the imaging device in the body cavity—e.g. relative to the cervix when used in colposcopy applications.

### BRIEF DESCRIPTION OF THE DRAWINGS

Embodiments of the present invention will now be described, by way of non-limiting example, with reference to the drawings in which:

FIG. 1 is a perspective view of a medical device in accordance with present teachings;

FIG. 2 is a top view of the medical device of FIG. 1;

FIG. 3 is a side view of the medical device of FIG. 1;

FIG. 4 is an end if you of the medical device of FIG. 1, looking from the distal end;

FIG. 5 is a partial, cross-sectional view of a medical device in accordance with present teachings, with the anchoring device in an expanded condition;

FIG. 6 is a partial, cross-sectional view of the medical device of FIG. 5 in a collapsed condition;

FIG. 7 is a top view of an alternative embodiment of a medical device in accordance with present teachings;

FIG. 8 is an alternative embodiment of a medical device in accordance with present teachings;

FIG. 9 is an alternative embodiment of a medical device in accordance with present teachings;

FIG. 10 is a view of the medical device of FIG. 9 with the casing removed;

FIG. 11 illustrates a system for imaging a body cavity, in accordance with present teachings;

FIG. 12 illustrates an alternative embodiment of a system for imaging a body cavity, in accordance with present teachings;

FIG. 13 shows the system of FIG. 12 in a stowed condition; and

FIG. 14 illustrates an alternative embodiment of a system for imaging a body cavity, in accordance with present teachings.

### DETAILED DESCRIPTION

Hereafter described are medical devices for imaging a body cavity and systems incorporating such devices. The medical devices disclosed herein have a reduced section size during insertion when compared with during imaging while inside the body cavity. This avoids the need for a speculum. Moreover, expansion of the device, particularly in anchoring device thereof, in the body cavity improves the accuracy and ease of self-examination of the service or other organ.

In some embodiments, a removable head is provided, enabling the head to be sterilised, washed or disposed of. The head and other components of the device may be made from any appropriate material, such as silicon.

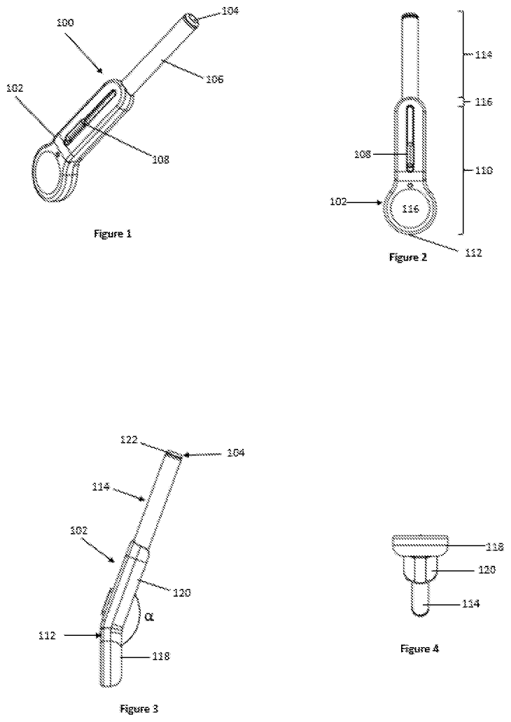

FIG. 1 illustrates medical device 100 providing the above advantages. The medical device 100 broadly includes an elongate body 102, and imaging device 104, and anchoring device 106 and a slider mechanism 108.

The medical device 100 in the embodiment shown is a colposcope. In other embodiments, the medical device may be configured as a proctoscope, rhinoscope or other device configured to examine part of the body of a subject (e.g. an organ, such as the cervix or rectum, of a patient) through a natural orifice.

The body 102 has a proximal region 110 positioned, in use, outside the body cavity. The length of the proximal region 110 from the proximal end 112 as shown in FIG. 2 is illustrative only. Moreover, the body 102 comprises a distal region 114 position, in use, inside the body cavity. The proximal region 110 may extend from the proximal end 112 to the distal region 114, or there may be a transition or other region 116 between the proximal region 110 and distal region 114.

The distal region 114 has a narrow cross-section during insertion, as shown in FIG. 2. This enables the distal region 114 to be comfortably inserted into the body cavity. Extending approximately from the distal region 114, the body 102 becomes slightly wider. While the widening is not essential, in the present embodiment that widening maintains integrity of the body 102 while enabling the slider 108 to be incorporated thereinto.

Towards the proximal end 112 of the proximal portion 110, the body 102 widens (see reference numeral 118). The widened portion 118 can be securely held in the palm of the user during actuation of the slider 108. As shown in FIG. 3, the widened portion 118 is at an angle α to a portion of the proximal region 110 between the widened portion 118 and the distal region 114. The angle α may be any desired angle, such as 130°, 145°, 160° or 180° (i.e. portions 118, 120 form a straight body). The angle α helps a user maintaining grip on the body 102 during use.

The widened portion 118 is also flat as shown in FIG. 4, with shows the transition in cross-sectional width between the widened portion 118, portion 120 and distal region 114. In other embodiments, the cross-sectional width variation may be different all portions of the body may have a common cross-sectional width.

The imaging device 104 is disposed at the distal region 114. Presently, the imaging device 104 is attached at, or forms, the distal tip 122. The imaging device 104 may be a camera or other imaging device. The imaging device 104 may also include a light source for illuminating a portion of the body cavity, such as the cervix or other portion located distally of the imaging device 104. The light source may be configured to emit a particular colour of light, or wavelength of light to preferentially illuminate particular types of tissue or a contrast medium. Similarly, the imaging device 104 may be configured to capture light of a particular colour or wavelength.

FIG. 6 is a partial view of an alternative medical device 200. The medical device 200 includes an imaging device 202 in a distal region 204. The imaging device 202, in the present instance, is immediately behind a distal tip 206 of the distal region 204.

The distal tip 206 may be configured to secure an anchoring device 208 to the body 210. The distal tip 206 may also, or alternatively, comprise or form a transparent lens through which the imaging device 202 can view a region inside body cavity that is distal of the distal tip 206. In some embodiments, the distal tip 206 may form part of the imaging device 202 and, in other embodiments, the distal tip 206 may instead form part of the body 210.

With reference to FIGS. 5 to 7, the anchoring device 208 has two opposed ends 212, 214. The anchoring device 208 is movable between a collapsed condition as shown in FIG. 6, and an expanded condition as shown in FIGS. 5 and 7. The collapsed condition facilitates entry of the imaging device 202 into the body cavity and, in the expanded condition, the anchoring device 208 engages (e.g. contacts or parts) an internal wall of the body cavity.

The two opposed ends 212, 214 of the anchoring device 208 are a proximal end (214) and a distal end (212). The distal end 212 is attached at the distal region 204 to the elongate body 210. Attachment is via a tongue or rib and groove arrangement. The tongue or rib 216 is formed on the body 210, with the groove 218 being formed in the anchoring device 208. In other embodiments, the tongue or rib may be formed in the anchoring device and the groove may be formed in the body. In other embodiments, the distal end 212 of the anchoring device 208 may be attached to the imaging device 202.

The proximal end 214 of the anchoring device 208 is attached to a slider 220. The proximal end 214 is movable, using the slider 220, towards the distal and 212. This moves the anchoring device 208 to the expanded condition as shown in FIGS. 5 and 7.

The anchoring device 208 may extend from the distal region 204 into the proximal region to connect to the slider 220, or the slider 220 may extend from the proximal region into the distal region 204 to connect to the anchoring device 208. Alternatively, the anchoring device 208 and slider 220 may me in any intermediate region between the proximal and distal regions of the body as mentioned with reference to FIG. 2.

The anchoring device 208 may move to the expanded condition by responding to forces generated in the anchoring device 208 by movement of the slider 220. Conversely, when the anchoring device 208 is in the expanded condition, the proximal and 214 of the anchoring device 208 may be moved away from the distal end 212 to move the anchoring device 208 collapsed condition.

To achieve the desired bending, the anchoring device 208 may be made from any suitable material or piece of material—e.g. silicon. The anchoring device 208 has a length extending between the two opposed ends 212, 214 and when the two opposed ends 212, 214 are brought towards each other, such that the distance between those ends is less than the length, the anchoring device 208 bends or buckles. Since, when in the collapsed condition, an internal wall 222 of the anchoring device 208 conforms with an external wall 224 of the body 210, the anchoring device 208 preferentially bends or buckles laterally outwardly with respect to an axis 226 the body 210.

The bending or buckling (these terms and similar being used interchangeably) occurs under stresses created in the piece of material forming the anchoring device 208. To that end, the anchoring device 208 may be formed from a single piece of material that encloses or ensheaths at least the distal region 204. In such embodiments, the anchoring device 208 may be considered to comprise a sheath around the distal region 204, extending from the distal region in a proximal direction.

The anchoring device 208 expands by bending in a mid-portion 228 between the two opposed ends 212, 214. With reference to FIG. 5, when the anchoring device 208 moves from the collapsed condition to the expanded condition the mid-portion 228 moves from a position proximal of the distal tip 206 of the body 210 to position distal of the distal tip 206. In so doing, the mid-portion 228 can be brought against an internal wall of the body cavity to stably maintain a position of the imaging device 202 relative to the internal wall. Since the mid-portion 228 projects distally of the distal tip 206, the imaging device 202 is spaced from the internal wall (generally designated by broken line 230).

The anchoring device 208 may be formed from a material having sufficient flexibility to allow the mid-portion 228 to remain in contact with the internal wall 230 while the body 210 is rotated, by a small amount, about an axis perpendicular to axis 226. This enables the field of view of the imaging device 202 to be redirected by a small amount without requiring repositioning of the anchoring device 208 on the internal wall 230.

With reference to the medical device 300 shown in FIG. 7, the anchoring device 302 is expanded laterally relative to an axis 304 of the body 306 in the expanded condition. While the anchoring device 302 may have a single expanded condition, it may instead have a plurality of expanded conditions each of which has a different amount of lateral expansion relative to the axis 304. For example, in the embodiment shown, the anchoring device 302 expands concentrically with respect to axis 304. For a first expanded condition of the anchoring device 302, the diameter of the anchoring device 302 (i.e. the width at its widest point) may be that indicated by reference numeral 308, and in a second expanded condition of the anchoring device 302, the diameter of the anchoring device 302 may be that indicated by reference numeral 310. This enables the degree of expansion of the anchoring device 302 when in the expanded condition to be selected to suit the size of the body cavity, the desired distance between the imaging device and the internal wall of the body cavity, or the required field of view.

The plurality of expanded conditions of the anchoring device 302 of the present embodiment are continuous—i.e. the slider 312 may be stopped at any location along its run 314. In other embodiments, the plurality of expanded conditions are discrete—e.g. the slider may be stopped at a defined number of locations along its run using any appropriate known arrangement.

The slider mechanism 312 extends from the proximal region 316 to the anchoring device 302. The slider 312 is movable to cause relative movement between the two opposed ends of the anchoring device (e.g. ends 212, 214 of the embodiment in FIG. 6), thereby to move the anchoring device 302 between the collapsed condition and expanded condition. The slider mechanism 312 includes an actuator 318 at the proximal region 316. The actuator is actuated directly by a finger or hand of the user, to move the slider and thereby control expansion and collapse of the anchoring device 302.

In the embodiment shown in FIG. 7, a power source (not shown) is provided in the body 306 for powering the imaging device (not shown). The power source also empowers a transmitter (not shown) that is in communication with the imaging device and transmits images captured by the imaging device to a remote system. While some embodiments of the medical device may be connected directly to the remote system by physical wiring, the transmitter of the present device 300 is configured to wirelessly transmit images.

The medical device 300 also includes a light, presently light emitting diode (LED) 320, that indicates when power is being supplied to the imaging device—i.e. when device 300 is ON or OFF.

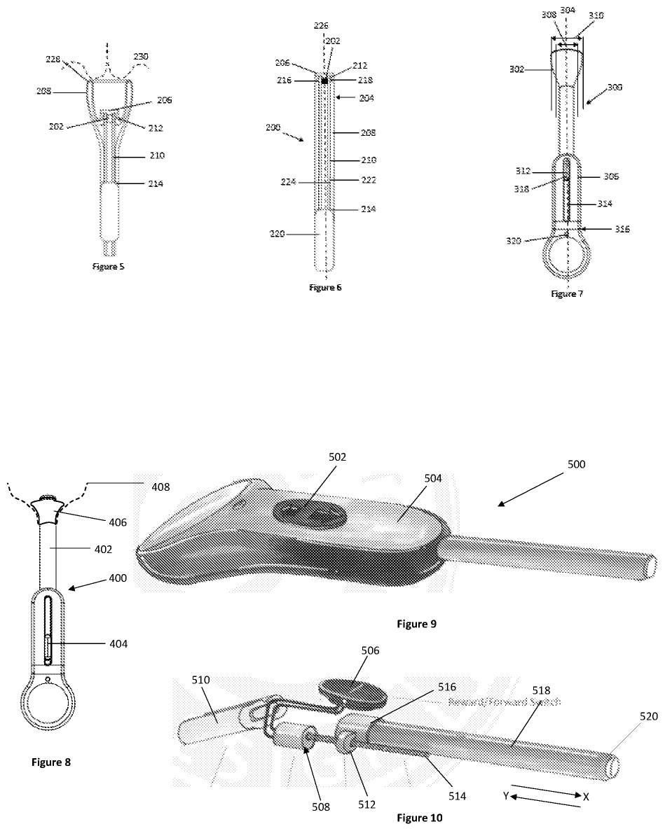

FIG. 8 shows a further medical device 400. The medical device includes similar components to medical devices 100, 200 and 300 of FIGS. 1 to 7, with the exception that the anchoring device 402 is secured at a proximal end to the slider 404 but is not connected at its distal end. When the slider 404 is advanced in the distal direction, the anchoring device 402 is stretched from the collapsed condition as shown in solid lines, over a fluted distal end 406 two and expanded condition 408 as shown in broken lines.

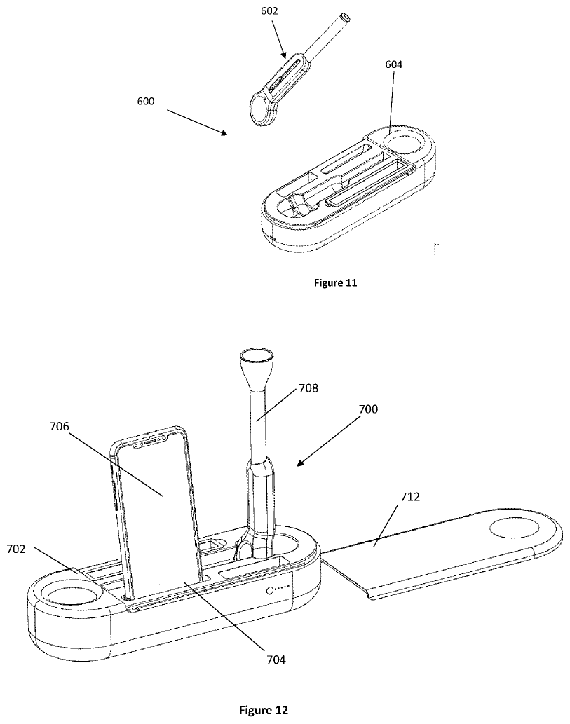

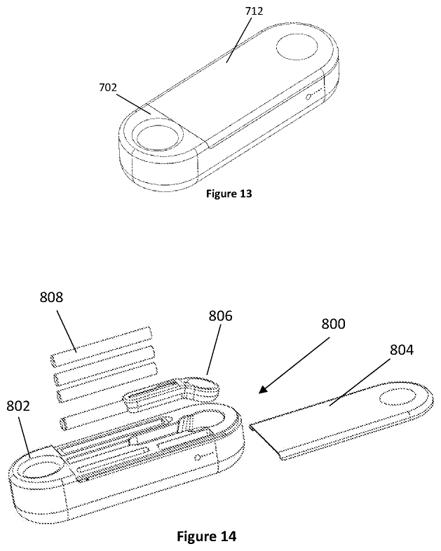

FIG. 9 shows a further embodiment of a medical device 500 in accordance with the present teachings. The device is similar in many respects with devices 100, 200, 300 and 400, with the exception that the slider 502 is electronic rather than manual. With reference to FIG. 10, in which housing 504 has been removed to expose the inner workings of the device 500, the slider 502 includes a button 506 that actuates a worm drive 508 it is powered by battery 510 to advance a threaded lug 512 along the worm drive shaft 514. The lug 512 is connected to the proximal end 516 of anchoring device 518 which advances towards the distal end 520 of the anchoring device 518 in the direction of arrow X, to move the anchoring device 518 to the expanded condition. Conversely, when the lug is moved in direction Y, the anchoring device 518 moves to the collapsed condition.

FIG. 11 shows a system 600 for imaging a body cavity. The system 600 includes a medical device 602, being a device according to one of embodiments 100, 200, 300, 400 and 500, or otherwise in accordance with present teachings. The system 600 includes a housing 604 and a receiver (not shown) at (e.g. housed within) the housing 604, and the power source (not shown) for powering the receiver. When in use, the medical device 602 and housing 604 of the present embodiment are physically separate, and the transmitter of a medical device 600 and transmits images wirelessly to the receiver. In other embodiments, the housing may have a wired connection to the medical device. The function and operation of the transmitter and receiver will be apparent to the skilled person in view of the present teachings.

The housing 604 is used to house the medical device 602 and associated components when not in use. To that end, the housing 604 includes various slots for receiving the medical device 602 and associated components.

With reference to the system 700 of FIG. 12, the housing 702 may comprise a dock 704 for engaging or receiving a portable device 706. The receiver, on receiving images from the transmitter of the medical device 708, may relay those images to the portable device 706. The receiver may receive the images wirelessly or may instead download the images from the medical device 708 when a medical device is in the housing, as shown, or stowed in the housing (i.e. in the slot 710 shaped to conform to the medical device 708). The receiver may store the images prior to relaying them to the portable device 706, or may relay the images to the portable device 706 without prior storage of those images. Notably, when used herein, the word images can comprise a single image transmitted on the first occasion, and another one or more images transmitted on a second occasion.

The housing 702 further includes a lid 712 the slides over a component storage compartment to contain the medical device 708 and associated components in the housing 702 when not in use.

Finally, FIG. 14 illustrates shows a system 800, with housing 802 having lid 804 removed, a medical device 806 in accordance with present teachings and a plurality of replacement anchoring devices 808. The anchoring devices can be conveniently slid over the distal region of the medical device to engage the distal region by friction fit or other securement mechanism. The anchoring devices can similarly be conveniently removed by sliding off the distal region and can thereafter be washed, sterilised or discarded.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A medical device comprising:
an elongate body having a proximal region positioned, in use, outside a body cavity, and a distal region positioned, in use, inside the body cavity;
an imaging device at the distal region;
an anchoring device having two opposed ends and being movable between a collapsed condition to facilitate entry of the imaging device into the body cavity and an expanded condition for engaging an internal wall of the body cavity; and
a slider mechanism extending from the proximal region to the anchoring device and being movable to cause relative movement between the two opposed ends of the anchoring device, thereby to move the anchoring device between the collapsed condition and expanded condition,
wherein the two opposed ends of the anchoring device are a proximal end and a distal end, the distal end being attached at the distal region to the imaging device, wherein the proximal end is movable towards the distal end to move the anchoring device to the expanded condition.

2. The medical device of claim 1, wherein the medical device is a colposcope.

3. The medical device of claim 1, wherein the slider mechanism comprises an actuator at the proximal region.

4. The medical device of claim 1, wherein the distal end is attached at the distal region to the elongate body.

5. (canceled)

6. The medical device of claim 1, wherein, when the anchoring device is in the expanded condition, the proximal end is movable away from distal end to move the anchoring device to the collapsed condition.

7. The medical device of claim 1, wherein the anchoring device comprises a sheath.

8. The medical device of claim 7, wherein the sheath extends from the distal region in a proximal direction.

9. The medical device of claim 7, wherein the sheath is formed from a piece of material and has a length, and when a distance between the two opposed ends is less than the length, the sheath bends between the two opposed ends under stresses created in the piece of material.

10. The medical device of claim 1, wherein the anchoring device is configured to expand by bending in a mid-portion between the two opposed ends.

11. The medical device of claim 10, wherein, when the anchoring device moves from the collapsed condition to the expanded condition, the mid-portion moves from a position proximal of a distal tip of the elongate body to a position distal of the distal tip.

12. The medical device of claim 1, wherein the elongate body has an axis extending between the proximal region and the distal region, and the anchoring device is expanded laterally relative to the axis in the expanded condition.

13. The medical device of claim 12, wherein the anchoring device has a plurality of expanded conditions each having a different amount of lateral expansion relative to the axis.

14. The medical device of claim 13, wherein the plurality of expanded conditions are discrete.

15. The medical device of claim 13, wherein the plurality of expanded conditions are continuous.

16. The medical device of claim 1, comprising a transmitter in communication with the imaging device, for transmitting images captured by the imaging device to a remote system.

17. A system for imaging a body cavity, comprising:
a medical device according to claim 16;
a housing;
a receiver at the housing; and
a power source for powering the receiver,
wherein, in use, the transmitter transmits images to the receiver.

18. The system of claim 17, wherein, in use, the medical device and housing are physically separate, and the transmitter wirelessly transmits images to the receiver.

19. The system of claim 17, wherein the housing comprises a dock for engaging a portable device, the receiver relaying the images to the portable device.
